(19) United States
(12) Patent Application Publication
(10) Pub. No.: US 2013/0115691 A1
(43) Pub. Date: May 9, 2013

(54) THERMO-CONDUCTIVE CELL CULTURE DISH HOLDER

(71) Applicant: Biocision, LLC, Mill Valley, CA (US)

(72) Inventor: Brian Schryver, Redwood City, CA (US)

(73) Assignee: BIOCISION, LLC, Mill Valley, CA (US)

(21) Appl. No.: 13/671,393

(22) Filed: Nov. 7, 2012

Related U.S. Application Data
(60) Provisional application No. 61/556,703, filed on Nov. 7, 2011.

(51) Int. Cl. C12M 1/00

(52) U.S. Cl. CPC C12M 1/005
USPC 435/303.1

(57) ABSTRACT

The present invention describes various devices for holding cell culture dishes in a secure manner and to ensure rapid and uniform heat transfer to and from the cell culture dish.

### RELATED APPLICATIONS

This application claims priority to U.S. Provisional Patent Application Ser. No. 61/556,703, filed on Nov. 7, 2011 and entitled THERMO-CONDUCTIVE CELL CULTURE DISH HOLDER, which is incorporated herein in its entirety.

### BACKGROUND OF THE INVENTION

Cell culture dishes are used frequently in research and medical laboratories. In many of these applications, there is a need to handle a cell culture dish while at the same time controlling its temperature.

One example of such use is the harvesting of cellular lysates of cell monolayers. Cell culture dishes are commonly used to culture live cells that require surface attachment for growth. To harvest a cell monolayer as cellular lysate, a lytic buffer containing detergents may be applied directly to the adherent cell monolayer in a cell culture dish. The insoluble portion of the cellular monolayer and lysate solution are then recovered in a process involving mechanical scraping of the dish surface using a plastic or rubber blade. During this process, the culture dish and its contents need to be cooled to near zero degrees Celsius to reduce thermally-induced changes in the molecular components of the cellular lysate.

A second example of such use is in the performance of attachment-independent growth assays. Anchorage-independent growth is one indicator of the tumorigenic potential of a cell. In this type of assay, cells are suspended in an agar solution with growth media, and the suspension is plated onto cell culture dishes. It is of critical importance in this assay that the cell culture dishes are cooled during or immediately after plating so that the agar solidifies rapidly, preventing cells from settling onto the culture dish surface.

A third example of such use is in tissue dissections. Chilling during dissection greatly enhances the preservation of tissue, cellular, and molecular structures.

In addition to the uses exemplified above, there are numerous other applications, including but not limited to cell growth, cell processing, cell analysis, and cell and enzyme assaying, in which cell culture dishes need to be cooled or warmed rapidly and/or while concomitantly being handled. This is, however, difficult to achieve given the current practices for controlling the temperature of cell culture dishes.

For cooling, current practice typically involves placing the culture dish on ice, in a refrigerator, or in a freezer. For warming, current practice typically involves placing the culture dish in a water bath, in an incubator, or in an oven. All of these practices involve either a larger, closed apparatus (e.g., refrigerator, freezer, incubator, oven), which complicates rapid, local temperature control and concomitant handling of the cell culture dish, or an unsteady surface (e.g., ice, ice/water bath), which is often also not conducive to the required handling.

For example, rapid cooling of a cell culture dish as required for the attachment-independent growth assay is difficult to achieve when the cell has to be moved from the inside of a sterile culture hood to a refrigerator. Similarly, the scraping process during harvesting of cellular lysates requires downward pressure on the cell culture dish surface, as may the dissection of tissues, and this downward pressure when applied to a cell culture dish on ice may cause the crushed ice bed to yield under pressure, leading to the cell culture dish being upended or forcefully shifted, and potentially resulting in contamination and/or loss of the cellular lysate or dissected tissue.

To facilitate experimentation and processing of samples in cell culture dishes, devices are needed that provide rapid and local means for controlling the temperature of cell culture dishes, and stable platforms for their handling. The present invention provides such devices and methods for their use.

### BRIEF DESCRIPTION OF THE DRAWINGS

FIG. 1 is a view of a thermo-conductive device of the invention for cooling or warming of a single circular cell culture dish having a diameter of 100 mm.

FIG. 2 is a view of a thermo-conductive device of the invention for cooling or warming of any of four different sizes of circular cell culture dishes having diameters of 35 mm, 60 mm, 100 mm, or 150 mm.

FIG. 3 is a cross section view of a portion of the device shown in FIG. 1.

FIG. 4 is a top view line drawing of the device shown in FIG. 2.

### DETAILED DESCRIPTION OF THE INVENTION

Provided herein are devices for the stable positioning and rapid, uniform, and local cooling and/or warming of cell culture dishes.

In one aspect, provided herein is a thermo-conductive device for cooling or warming of a cell culture dish, wherein the device comprises a top surface, wherein the top surface comprises one or more recessed areas, wherein the recessed areas engage one or more projections present on an underside of the cell culture dish, and wherein engagement of the one or more recessed areas and the one or more projections stabilizes the cell culture dish on the top surface of the thermo-conductive device.

The term “cell culture dish” as used herein refers to a dish that holds any of a variety of samples in a laboratory (e.g., a cell suspension, a tissue sample, an assay mixture). The cell culture dish comprises a bottom container with a substantially flat bottom wall in which the sample may be placed. The cell culture dish may further comprise a lid with which the bottom container may be covered. The cell culture dish may be made of any material, including but not limited to polystyrene or glass. The cell culture dish may have any shape, including but not limited to circular, square, triangular, and rectangular, and may be of any size suitable for use in the laboratory. In some embodiments, the cell culture dish is circular and has a diameter selected from the group consisting of diameters of 35 mm, 50 mm, 60 mm, 80.5 mm, 92 mm, 100 mm, and 150 mm. The cell culture dish may lack any vertical divisions inside the bottom container and thus provide only a single compartment for sample placement. Alternatively, the cell culture dish may comprise one or more vertical divisions inside the bottom container and thus provide multiple compartments for sample placement.

In some embodiments, the cell culture dish may comprise a coating (e.g., a collagen, poly-D-lysine, or gelatin coating) on the bottom wall inside the bottom container to facilitate attachment of sample components (e.g., cells). In other embodiments, the cell culture dish does not comprise such a coating, and is more suitably used when attachment of sample components is not desired or not needed (e.g., to determine the oncogenic potential of transformed cells in a attachment-independent growth assay, to grow microbial colonies on agar, or to dissect a tissue sample), or when specific coating is to be applied to effect attachment of specific sample components (e.g., an antibody coating to effect attachment of specific antigens). Cell culture dishes that do not comprise coating are commonly referred to as “Petri dishes”.

Cell culture dishes are commercially available, for example, from Sigma-Aldrich (St. Louis, Mo.; Corning plastic Petri and culture dishes with product numbers CLS3294, CLS3295, CLS3296, CLS3260, CLS430589, CLS430591, CLS430597, CLS430588, CLS430165, CLS430166, CLS430196, CLS430599, CLS430293, CLS430167, CLS3261, CLS3262), Sarstedt (Nuembrecht, Germany; lummox cell culture dishes with product numbers 94.6077.305, 94.6077.331, and 94.6077.410; Petri dishes with product numbers 82.1135, 82.1184, 82.1194, 82.1195, and 82.1472), and Becton, Dickinson & Co. (Franklin Lakes, N.J.; product numbers 351003, 351005, 351006, 351007, 351008, 351009, 351013, 351016, 35129, 351058, 354550). Most of these cell culture dishes are circular in shape, but non-circular cell culture dishes (e.g., rectangular or square dishes) are also commercially available, for example, from Sigma-Aldrich (St. Louis, Mo.; Corning bioassay dishes with product numbers CLS431110, CLS431111, CLS431272, and CLS431301), Sarstedt (Nuembrecht, Germany; quadriPERM dish with product number 94.6077.307), and Becton, Dickinson & Co. (Franklin Lakes, N.J.; product numbers 351040 and 351112). The devices of the invention include those that accommodate circular and those that accommodate non-circular cell culture dishes.

Many commercially available cell culture dishes comprise ring-shaped ridges on the undersurface of their bottom containers to protect the undersurface from scratching, and to facilitate stacking of the cell culture dishes by engaging a counterpart ring on the lid of the subordinate cell culture dish in the stack.

The devices of the invention have many different applications. Some such applications involve rapid and uniform cooling of a cell culture dish. Other such applications involve rapid and uniform warming of a cell culture dish. Yet other such applications involve maintaining the temperature of a cell culture dish. Further, some applications include sample preparation, biopsies, and immunohistochemistry procedures. These multiple applications are herein referred to collectively as “cooling or warming”. One important aspect of the devices of the invention is that, due to their construction from highly thermo-conductive material, they ensure that heat transfer to and from the cell culture dish is rapid.

Another important aspect of some of the devices of the invention is that the top surface of the device—the surface in contact with the bottom surface of the cell culture dish—is of a size and shape to ensure maximum (complete and contiguous) contact between the top surface of the device and the bottom surface of the cell culture dish. This helps to ensure that heat transfer to and from the device is uniform across the entire bottom surface of the cell culture dish, ensuring that its contents are uniformly cooled or heated, minimizing anomalous results due to inconsistent cooling or heating of the contents of the dish.

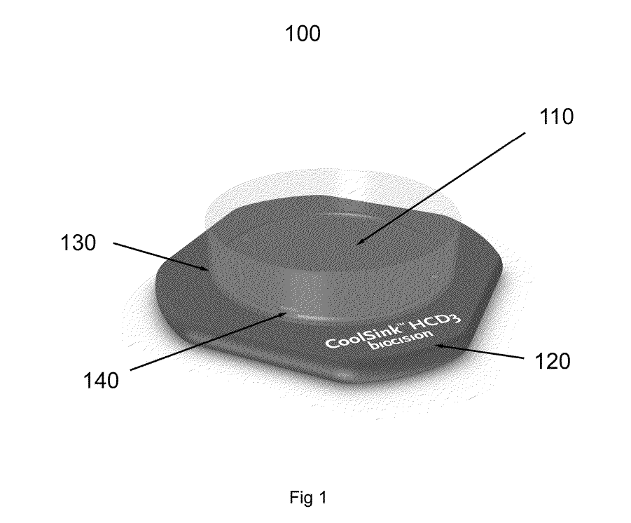

Embodiments of the invention will be described and their various features illustrated with reference to the drawings in FIGS. 1 through 4.

Referring to FIG. 1, an embodiment of the device is shown wherein device 100 is generally square but has rounded corners and beveled edges 120. The device is designed to engage at the surface plane 130 with a round cell culture dish 110 having a diameter of 100 mm. A recess channel 140 in the top surface of the device can receive and accommodate projecting features on the undersurface of the cell culture dish. The term “recess channel” as used herein refers to an indented groove on a surface.

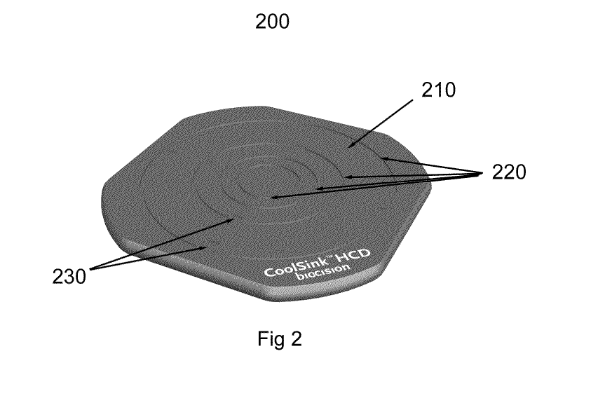

Referring to FIG. 2, a second embodiment of the device is shown wherein the device 200 comprises four recess channels 220 and various recess excavations 230 on the top surface 210 to receive and accommodate projecting features on the undersurface of any of four different circular cell culture dishes having diameters of 35 mm, 60 mm, 100 mm, or 150 mm. The term “recess excavation” as used herein refers to an indented area of variable shape and size on a surface.

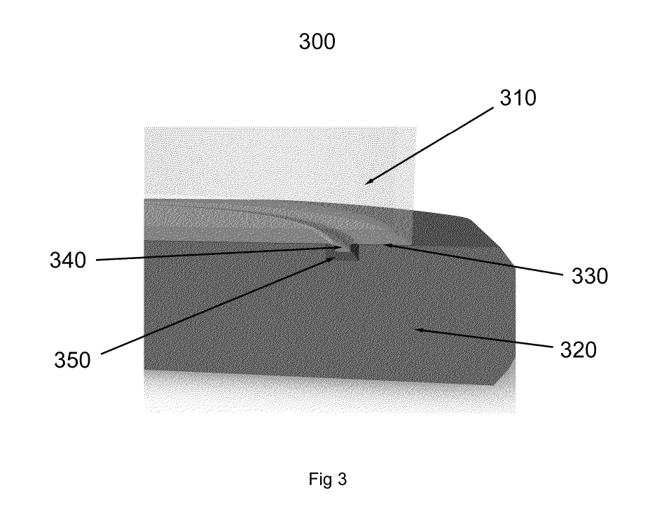

Referring to FIG. 3, a cross section rendering 300 of a portion of device 100 of FIG. 1 is shown. The device body 320 comprises a recess channel 350 on its top surface that provides clearance for the projecting ring 340 of the cell culture dish 310. The clearance provided allows direct contact of the cell culture dish undersurface with the top surface 330 of the device, thereby maximizing thermal energy transfer between the device and the cell culture dish.

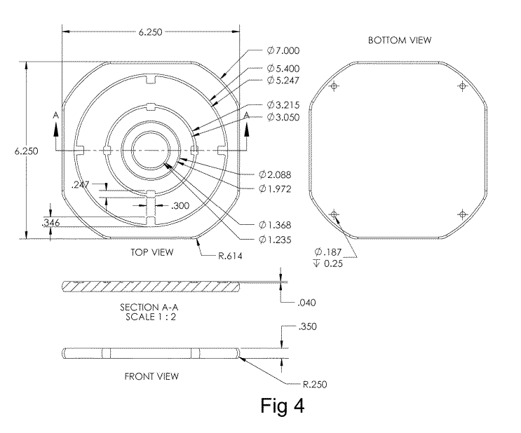

Referring to FIG. 4, a line drawing of the device in FIG. 2 is shown. The device has a width and length of 6.250 inches, and a thickness of 0.35 inches. In other embodiments, the width, length, and thickness of the device may have different dimensions, but generally will lie between 3 inches and 7 inches for width, between 3 inches and 7 inches for length, and between 0.25 inches and 0.5 inches for thickness.

The device is highly thermo-conductive by virtue of it comprising, consisting, or consisting essentially of a thermo-conductive material, such as a metal or a metal alloy. The term “thermo-conductive” as used herein refers to the ability to conduct thermal energy. Suitable thermo-conductive materials include but are not limited to aluminum (i.e., anodized aluminum), an aluminum alloy, copper, a copper alloy, and combinations thereof. In some embodiments, the thermo-conductive material has the capacity to rapidly adapt to any temperature from between −150° C. and +150° C., from between −100° C. and +100° C., from between −75° C. and +75° C., and from between −50° C. and +50° C. Suitable thermo-conductive material may also include materials that may be sterilized via autoclave, high heat, bleach, alcohol, or other lab disinfectants and detergents.

Referring again to FIG. 4, the top surface of the device comprises four concentric recess channels with inside/outside diameters of 1.235±0.005/1.368±0.005 inches, 1.972±0.005/2.088±0.005 inches, 3.050±0.005/3.215±0.005 inches, and 5.247±0.005/5.400±0.005 inches. Each recess channel has a depth of 0.04 inches. The two most peripheral recess channels each comprise four evenly spaced rectangular recess excavations with dimensions of 0.300 inches×0.247 inches for the second most peripheral recess channel, and 0.300 inches×0.346 inches for the most peripheral recess channel. The positions of the recess channels and recess excavations in this embodiment of the invention minor the shapes and positions of projections present on the undersurface of circular Corning culture dishes and circular Corning Petri dishes with diameters of 35 mm (Sigma-Aldrich product numbers CLS3294, CLS430588, and CLS430165), 60 mm (Sigma-Aldrich product numbers CLS3295, CLS430589, CLS430166, CLS430196, and CLS3261), 100 mm (Sigma-Aldrich product numbers CLS3296, CLS430591, CLS430167, and CLS3262), and 150 mm (Sigma-Aldrich product numbers CLS430597 and CLS430599). Placement of such Corning culture and Petri dishes on the device allows their projections to engage in the recess channels and bulges on the top surface of the device, thus providing full contact of the undersurface of the dish with the top surface of the device, thereby maximizing thermal energy transfer. The illustrated embodiment is therefore most suitably useful for the cooling or warming of the above listed Corning culture dishes and Corning Petri dishes.

Also within the scope of the present invention are embodiments in which the device has different dimensions (e.g., different widths, lengths, and/or thicknesses) than the devices shown in FIGS. 1 through 4, embodiments in which the device has a different shape (e.g., rectangular, circular, triangular) than the devices shown in FIGS. 1 through 4, and embodiments in which the device comprises more or fewer recess channels (e.g., one, two, three, five, six, seven, more than seven) and recess excavations than the devices shown in FIGS. 1 through 4, making the device most suitably useful for the cooling or warming of only some of the above listed Corning culture dishes and Corning Petri dishes.

In other embodiments, the device comprises recessed areas with different dimensions and shapes as the ones shown in FIGS. 1 through 4, making the top surface of the device able to engage projections on the undersurface of cell culture dishes other than those present on circular Corning culture and Petri dishes. Such other projections include but are not limited to raised rings, feet, letters, numbers, markings, symbols, structural ridges, mold sprues, flashings, and plastic tags. In some embodiments, the device is most suitably useful for the cooling or warming of cell culture dishes other than circular Corning culture and circular Petri dishes.

In some embodiments, the device further comprises on its underside foot structures that elevate the device from an underlying surface. The term “foot structures” as used herein refers to protruding structures. In some such embodiments, the foot structures are made of a thermally insulating material (e.g., rubber). In some such embodiments, the foot structures are optionally removable. Typically three, four, or more such structures will be present in embodiments including them. In some embodiments, the device further comprises on its underside a riser with which it can be fitted into a heating block.

In some embodiments, the devices of the invention are of a size sufficient to support more than one culture dish at a time, i.e., are capable of supporting 2, 3, 4 or more culture dishes at one time (on a single device).

In some embodiments, the devices of the invention further comprise a separate lid (thus constituting a device consisting of a base and a separate lid, although the lid may in some embodiments be connected to the base via a connecting means such as a hinge) that fits over the top of the cell culture dish (generally a lid is supplied with a cell culture dish, and the lid of the device engages and is in intimate contact with the lid of the dish) and is made of the same or a similar highly thermo-conductive material as the base that supports the dish.

The devices provided herein are useful in cooling or warming cell culture dishes. The cell culture dish is placed on the device such that the projections on the undersurface of the cell culture dish engage with at least some of the recessed areas present on the top surface of the device, thus steadying the position of the cell culture dish on the surface of the device. For cooling, the device is positioned on a cold surface of the desired temperature, such as, for example, ice, a cold plate, dry ice, liquid nitrogen, and other temperature sources. For warming, the device is positioned on a warm surface of the desired temperature, such as, for example, a hot plate.

The devices provided herein have several remarkable attributes. For one, the devices conduct thermal energy rapidly and evenly across their surfaces. Their thermo-conductivity coupled with the intimate contact between the top surfaces of the devices and the undersides of the cell culture dishes placed on them, which is enabled by the engagement of the recessed areas in the top surfaces of the devices and of the protruding features on the underside of the cell culture dishes, enables rapid and uniform cooling or warming of cell culture dishes across their entire surfaces. Such rapid and uniform cooling or warming can reduce variability in sample handling or analysis, which in turn can be critical to the outcome of experiments in basic and clinical research and testing.

In addition, the wider circumferences and weights of the devices compared to that of the cell culture dishes that are placed on them help stabilize the assemblies on cold or warm surfaces as compared to direct placement of the cell culture dishes on the cold or warm surfaces. Also, by eliminating direct contact between the cell culture dishes and the cold or warm surfaces, the devices protect the content of the cell culture dishes and reduce the risk for sample contamination. The devices are thus useful for all aspects of temperature-controlled handling of the contents of cell culture dishes, ranging from maintenance to processing to analysis to transport.

In another aspect, the present invention provides a method for rapidly and uniformly cooling a cell culture dish while harvesting a cellular lysate of a cell monolayer in the cell culture dish. In one embodiment, such method comprises the following steps:

a) placing the cell culture dish comprising the cell monolayer on the device;

b) placing the device on a cold surface (e.g., ice);

c) adding to the cell culture dish cold lytic buffer; and

d) recovering the cellular lysate.

Other embodiments comprise essentially these same steps except that the order of steps a), b), and/or c) may be changed. For example, the device may be placed on ice prior to placing the cell culture dish on the device, or cold lytic buffer may be added to the cell culture dish prior to placing the cell culture dish on the device or prior to placing the device on the cold surface.

In yet another aspect, the present invention provides a method for rapidly and uniformly cooling a cell culture dish comprising cells suspended in agar. In one embodiment, such method comprises the following steps:

a) placing the cell culture dish on the device;

b) placing the device on a cold surface (e.g., ice); and

c) adding cells suspended in agar to the cell culture dish.

Other embodiments comprise essentially these same steps except that the order of steps a), b), and/or c) may be changed. For example, the device may be placed on ice prior to placing the cell culture dish on the device, or cells suspended in agar may be added to the cell culture dish prior to placing the cell culture dish on the device or prior to placing the device on the cold surface.

In yet another aspect, the present invention provides a method for cooling a cell culture dish while dissecting a tissue sample. In one embodiment, such method comprises the following steps:

a) placing the cell culture dish on the device;

b) placing the device on a cold surface (e.g., ice);

c) placing the tissue sample in the cell culture dish; and

d) dissecting the tissue sample.

Other embodiments comprise essentially these same steps except that the order of steps a), b), and/or c) may be changed. For example, the device may be placed on ice prior to placing the cell culture dish on the device, or the tissue sample may be placed in the cell culture dish prior to placing the cell culture dish on the device or prior to placing the device on the cold surface.

1. A device for cooling or warming of a cell culture dish, wherein the device is thermo-conductive, wherein the device comprises a top surface and a bottom surface, wherein the top surface comprises one or more recessed areas, wherein the one or more recessed areas engage one or more projections present on an underside of the cell culture dish, and wherein engagement of the one or more recessed areas and the one or more projections stabilizes the cell culture dish on the top surface of the thermo-conductive device and provides an increased area of direct contact between the undersurface of the cell culture dish and the top surface of the device.

2. The device of claim 1, wherein the cell culture dish is a non-circular cell culture dish.

3. The device of claim 1, wherein the cell culture dish is a circular cell culture dish.

4. The device of claim 3, wherein the circular cell culture dish has a diameter selected from the group consisting of 35 mm, 50 mm, 60 mm, 80.5 mm, 92 mm, 100 mm, and 150 mm.

5. The device of claim 3, wherein the circular cell culture dish is a Corning culture dish.

6. The device of claim 3, wherein the circular cell culture dish is a Petri dish.

7. The device of claim 6, wherein the Petri dish is a Corning Petri dish.

8. The device of claim 1, wherein the device comprises a metal.

9. The device of claim 8, wherein the metal is selected from the group consisting of copper and aluminum.

10. The device of claim 1, wherein the device comprises a metal alloy.

11. The device of claim 10, wherein the metal alloy is selected from the group consisting of a copper alloy and an aluminum alloy.

12. The device of claim 1, wherein the one or more recessed areas comprise one or more ring-shaped recess channels.

13. The device of claim 12 wherein the one or more recessed areas further comprise one or more recess excavations.

14. The device of claim 13, wherein the device has a width and length of 6.250 inches, a thickness of 0.35 inches; wherein the top surface comprises four concentric recess channels with inside/outside diameters of 1.235±0.005/1.368±0.005 inches, 1.972±0.005/2.088±0.005 inches, 3.050±0.005/3.215±0.005 inches, and 5.247±0.005/5.400±0.005 inches; wherein each recess channel has a depth of 0.04 inches; and wherein the two most peripheral recess channels each comprise four evenly spaced rectangular bulges with dimensions of 0.300 inches×0.247 inches for the second most peripheral recess channel, and 0.300 inches×0.346 inches for the most peripheral recess channel.

15. The device of claim 1, wherein the bottom surface comprises three or more foot structures, and wherein the foot structures elevate the base from an underlying surface.

16. The device of claim 15, wherein the foot structures have low thermo-conductivity.
